SEQUENCE LISTING (4 sequences)

SEQ ID NO: 1 - DNA, 20 bp, synthetic construct
  misc_feature 1..20: note = The sequence is synthetized.
  source 1..20: mol_type = other DNA
aggagaccgccgtttttgat

SEQ ID NO: 2 - DNA, 20 bp, synthetic construct
  misc_feature 1..20: note = The sequence is synthetized.
  source 1..20: mol_type = other DNA
aagcgtttcgtttcggttgg

SEQ ID NO: 3 - DNA, 20 bp, synthetic construct
  misc_feature 1..20: note = The sequence is synthetized.
  source 1..20: mol_type = other DNA
ctatggctaccgcgacgtac

SEQ ID NO: 4 - DNA, 18 bp, synthetic construct
  misc_feature 1..18: note = The sequence is synthetized.
  source 1..18: mol_type = other DNA
gctgctcacgtcgacgac

(19) United States
(12) Patent Application Publication
(10) Pub. No.: US 2024/0175097 A1
(43) Pub. Date: May 30, 2024

(54) MOLECULAR MARKERS LINKED TO MAIN QTL FOR REGULATING RESISTANCE OF RICE TO WHITEBACKED PLANTHOPPER AND APPLICATION THEREOF

(71) Applicant: Zhejiang Normal University, Jinhua (CN)

(72) Inventors: Yuchun RAO, Jinhua (CN); Hanfei YE, Jinhua (CN); Yuexing WANG, Jinhua (CN); Tao LU, Jinhua (CN); Zhitao LIU, Jinhua (CN); Jiyi XIE, Jinhua (CN); Yuxin DAI, Jinhua (CN); Ziwen HUANG, Jinhua (CN)

(73) Assignee: Zhejiang Normal University, Jinhua (CN)

(21) Appl. No.: 18/342,759

(22) Filed: Jun. 28, 2023

Related U.S. Application Data
(63) Continuation of application No. PCT/CN2023/087070, filed on Apr. 7, 2023.

(30) Foreign Application Priority Data
Nov. 26, 2022 (CN) 202211491947.8

(51) Int. Cl. C12Q 1/6895

(52) U.S. Cl. CPC C12Q 1/6895; C12Q 2600/13

(57) ABSTRACT

Molecular markers linked to main QTL for regulating resistance of rice to whitebacked planthopper are provided. The QTL is located on rice chromosome 1, with a genetic distance of 30.88-34.32 cM and a physical distance of 7203302-8007653 bp. Then the molecular markers are set on both sides of the QTL, and the molecular markers are used to screen rice varieties resistant to whitebacked planthopper, which improves the selection efficiency of ideal plant types of rice.

### CROSS REFERENCE TO RELATED APPLICATIONS

This application is the continuation application of International Application No. PCT/CN2023/087070, filed on Apr. 7, 2023, which is based upon and claims priority to Chinese Patent Application No. 202211491947.8, filed on Nov. 26, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBSJBJ002_Sequence_Listing.xml, created on Jun. 19, 2023, and is 6,011 bytes in size.

### TECHNICAL FIELD

The present disclosure relates to the technical field of molecular markers for rice whitebacked planthopper, and more specifically, to molecular markers linked to main QTL for regulating resistance of rice to whitebacked planthopper and application thereof.

### BACKGROUND

Rice is one of the world's most important food crops, and its safe production is of great importance to ensure food security. However, rice production is threatened by various plant diseases and insect pests all year round. Among them, whitebacked planthopper is widely distributed in East Asia, South Asia and Southeast Asia, and is one of the major pests of rice. As a widespread migratory pest, its occurrence is characterized by suddenness and outbreaks, which makes it more difficult to control the whitebacked planthopper. The whitebacked planthopper is a piercing-sucking pest that sucks the sap from the bast of rice stalks through oral needles, resulting in slowed plant growth, yellowing of leaves and, in severe cases, withering and death of the entire plant; in addition, the whitebacked planthopper is also a vector for the southern rice black streak dwarf virus (SRBSDV), a viral disease for which there is no cure and which is known as the “rice cancer”. It is a serious threat to rice production in East Asia and Southeast Asia.

Currently, the use of chemical pesticides is the main measure to control the whitebacked planthopper. However, the excessive use of chemical pesticides not only increases production costs, pollutes the environment and produces pesticide residues, but also kills natural enemies, destroys the ecological balance and leads to the development of pest resistance, causing the pest to become rampant again. Therefore, the breeding and promotion of resistant varieties is considered to be the most economical and effective measure to control whitebacked planthopper. The discovery of antigen and resistance genes of whitebacked planthopper is the prerequisite and basis for breeding resistant varieties. So far, multiple genes or QTL resistant to whitebacked planthopper have been reported. However, only qWL6 and qWBPHI1 have been precisely mapped, and there has been no report of map-based cloning of resistance genes to whitebacked planthopper in rice, and the mechanism of resistance of rice to whitebacked planthopper is still poorly understood.

Therefore, the development of new molecular markers linked to QTL for resistance of rice to whitebacked planthopper and its further application for the selection of superior rice varieties is an urgent problem for those in the field.

### SUMMARY

In view of the above, the present disclosure provides new molecular markers linked to QTL for resistance of rice to whitebacked planthopper, with which the selection and breeding of the rice resistant to whitebacked planthopper can improve rice breeding efficiency.

In order to achieve the above purpose, technical solutions of the present disclosure are specifically described as follows.

Molecular markers linked to main QTL for regulating resistance of rice to whitebacked planthopper are provided, and the rice molecular markers include Indel Wbph-1 and Indel Wbph-2;
wherein, primer pairs for the molecular marker Indel Wbph-1 are:

upstream primer:
SEQ ID NO: 1
5′-AGGAGACCGCCGTTTTTGAT-3′,;
downstream primer:
SEQ ID NO: 2
5′-AAGCGTTTCGTTTCGGTTGG-3′,;

primer pairs for the molecular the marker Indel Wbph-2 are:

upstream primer:
SEQ ID NO: 3
5′-CTATGGCTACCGCGACGTAC-3′,;
downstream primer:
SEQ ID NO: 4
5′-GCTGCTCACGTCGACGAC-3′,.

As the same inventive concept as the above technical solution, the present disclosure also requests the protection of the application of the molecular markers linked to main QTL for regulating resistance of rice to whitebacked planthopper in selection and breeding of rice varieties resistant to whitebacked planthopper.

As the same inventive concept as the above technical solution, the present disclosure also requests the protection of a method for selection and breeding of rice varieties resistant to whitebacked planthopper, including:
extracting rice DNA, using the primer pairs of the molecular markers of claim 1 for PCR amplification of the DNA to obtain amplification products, carrying out electrophoresis detection on the amplification products, and analyzing the resistance of rice to whitebacked planthopper by band pattern.

Preferably, the reaction system of the PCR amplification is: 1 μL of upstream primer, 1 μL of downstream primer, 2 μL of DNA template, 6 μL of mix enzyme, 1 μL of ddH2O; and the reaction procedure of the PCR amplification is:
pre-denaturation at 94° C. for 3 min; denaturation at 94° C. for 30 s, annealing at 57° C. for 30 s, extension at 72° C. for 30 s, amplification for 38 cycles; and finally, final extension at 72° C. for 10 min.

As the same inventive concept as the above technical solution, the present disclosure also requests the protection of a kit for selection and breeding of rice varieties resistant to whitebacked planthopper, which includes the primer pairs of the molecular markers.

According to the above technical solutions, compared with the prior art, the main QTL for regulating the resistance of rice to whitebacked planthopper is located on rice chromosome 1, with a genetic distance of 30.88-34.32 cM and a physical distance of 7203302-8007653 bp, and two pairs of molecular markers closely linked to the QTL locus are obtained, and the molecular markers can be adopted to predict the resistance of rice to whitebacked planthopper and speed up the breeding of the ideal plant type of rice.

### BRIEF DESCRIPTION OF THE DRAWINGS

In order to explain the embodiments of the present disclosure or the technical solutions in the prior art more clearly, the following drawings that need to be used in the description of the embodiments or the prior art are briefly introduced. Obviously, the drawings in the following description are only embodiments of the present disclosure. For those of ordinary skill in the art, other drawings can be obtained based on the drawings disclosed without creative work.

FIG. 1 shows the construction flow chart of genetic materials used in the process of locating the main QTL for regulating the resistance of rice to whitebacked planthopper;

FIG. 2 shows the frequency distribution of resistance of whitebacked planthopper in RIL population; wherein, Nekken represents rice variety Nekken 2, HZ represents rice variety HuaZhan;

FIG. 3 shows the location of the main QTL qWBPH1.1 on chromosome 1, which regulates the resistance of rice to whitebacked planthopper; wherein, 1 is HZ, 2 is Nekken 2, 3 is Nekken 2/HZ cross progeny F1, and 4-12 are the rice strain materials with higher resistance in RIL population of Nekken 2/HZ cross combination;

FIG. 4 shows the electropherogram generated by amplification of primer pairs of molecular marker Indel Wbph-1 in parents, their F1 generation and RIL populations; wherein, 1 is HZ, 2 is Nekken 2, 3 is Nekken 2/HZ cross progeny F1 (resistance biased male parent HZ), and 4-12 are the rice strain materials with higher resistance in RIL population of Nekken 2/HZ cross combination;

FIG. 5 shows the electropherogram generated by amplification of primer pairs of molecular marker Indel Wbph-2 in parents, their F1 generation and RIL populations;

FIG. 6 shows the electropherogram generated by amplification of primer pairs of Indel Wbph-1 in the parents HZ, Nipponbare and their F1 generation and BC₃F₁ generation with Nipponbare as recurrent parents; wherein, 1 is HZ, 2 is Nipponbare, 3 is HZ/Nipponbare cross progeny F1, and 4-10 are the electropherogram generated by amplification in BC₃F₁ generation with Nipponbare as recurrent parents;

FIG. 7 shows the electropherogram generated by amplification of primer pairs of Indel Wbph-2 in the parents HZ, Nipponbare and their F1 generation and BC₃F₁ generation with Nipponbare as recurrent parents; wherein, 1 is HZ, 2 is Nipponbare, 3 is HZ/Nipponbare cross progeny F1, and 4-10 are the electropherogram generated by amplification in BC₃F₁ generation with Nipponbare as recurrent parents;

### DETAILED DESCRIPTION OF THE EMBODIMENTS

Technical solutions in the embodiments of the present disclosure will be clearly and completely described below with reference to the accompanying drawings in the embodiments. Obviously, the described embodiments are only part of the embodiments of the present disclosure, not all of them. Based on the embodiments of the disclosure, all other embodiments made by those skilled in the art without sparing any creative effort should fall within the protection scope of the disclosure.

Embodiment 1 Locating of Main QTL for Regulating the Resistance of Rice to Whitebacked Planthopper

1. Acquisition of Experimental Materials

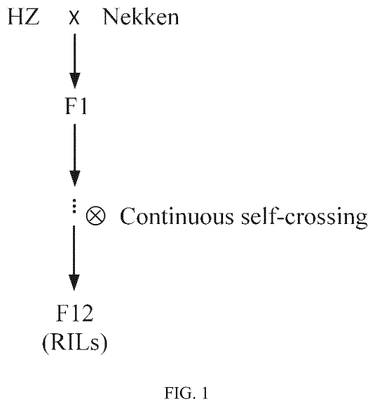

The crossing was carried out with Nekken 2 as the donor parent and the rice variety HZ as the recipient parent, and 120 genetically stable strains (F12, all strains were phenotypically stable) were finally obtained by the single seed descent method (i.e., F1 was treated with bagging and single plant inoculation until the phenotype of the progeny strains did not segregate) to form the recombinant self-crossing RIL population, as shown in FIG. 1.

60 seeds of the parents and each strain (F12) were selected. After surface disinfection, seeds were soaked for 2 days, wrapped with wet towels, and placed in a 37° C. incubator for germination for 48 hours. Seeds with the same germination degree were selected for sowing. After 30 days, 24 seedlings of the parents and each strain with similar growth conditions were selected for transplanting. All rice materials were planted in the experimental field of College of Biochemistry, Zhejiang Normal University, Jinhua City, Zhejiang Province, under routine management.

2. Data Determination of Resistance to Whitebacked Planthopper

The standard seed-box screening test (SSST) method was adopted to identify the resistance of rice to whitebacked planthopper.

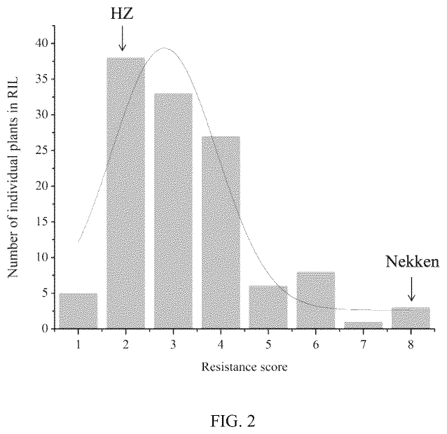

The results are as shown in FIG. 2, the data of resistance to whitebacked planthopper showed continuous normal distribution and a wide range, with many transgressive individuals (grade 1, high resistance), showing the genetic characteristics of quantitative traits.

3. QTL Locating Analysis

Using the genetic map constructed by a large number of SNPs and Indel markers developed in the early stage of the laboratory, the quantitative trait locus (QTL) interval mapping of resistance of rice to whitebacked planthopper was carried out. The relationship between the markers of the whole chromosome set and the phenotypic value of quantitative traits was analyzed by R-QTL professional software, QTL were mapped to the corresponding locations of the linkage group one by one, and their genetic effects were estimated. If molecular markers with LOD>2.5 were detected, it was considered that there was a QTL between the two markers corresponding to the highest LOD value.

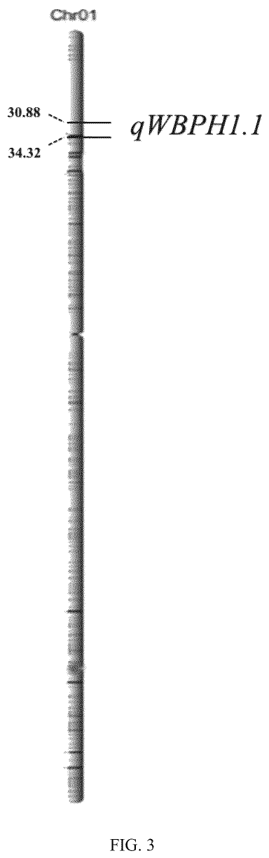

Finally, a main QTL between Indel Wbph-1 marker and Indel Wbph-2 marker on chromosome 3 was found in the whole genome of rice HZ. The LOD value of the resistance to whitebacked planthopper was as high as 3.32, with a genetic distance of 30.88-34.32 cM, a physical distance of 7203302-8007653 bp, and was named qWBPH1.1 (FIG. 3).

Embodiment 2 Molecular Marker Assisted Selection

Molecular markers Indel Wbph-1 and Indel Wbph-2 were set at the upstream and downstream of QTL locus qWBPH1.1, and primers were designed;
the primer pairs for the molecular marker Indel Wbph-1 were:

upstream primer:
SEQ ID NO: 1
5′-AGGAGACCGCCGTTTTTGAT-3′,;
downstream primer:
SEQ ID NO: 2
5′-AAGCGTTTCGTTTCGGTTGG-3′,;

the primer pairs for the molecular marker Indel Wbph-2 were:

upstream primer:
SEQ ID NO: 3
5′-CTATGGCTACCGCGACGTAC-3′,;
downstream primer:
SEQ ID NO: 4
5′-GCTGCTCACGTCGACGAC-3′,.

Genomic DNA was extracted from the leaves of the parents Nekken 2, HZ and their F1 generations and RIL populations, and PCR amplification was performed on their genomic DNA using the above molecular markers;

PCR reaction system: 1 μL of upstream primer (10 μmol), 1 μL of downstream primer (10 μmol), 2 μL of DNA template (>100 ng/μL), 6 μL of mix enzyme (TsingkeBiotechnologyCo., Ltd., 2×Taq Master Mix), 1 μL of ddH₂O;
reaction procedure: pre-denaturation at 94° C. for 3 min; denaturation at 94° C. for 30 s, annealing at 57° C. for 30 s, extension at 72° C. for 30 s, amplification for 38 cycles; and finally final extension at 72° C. for 10 min.

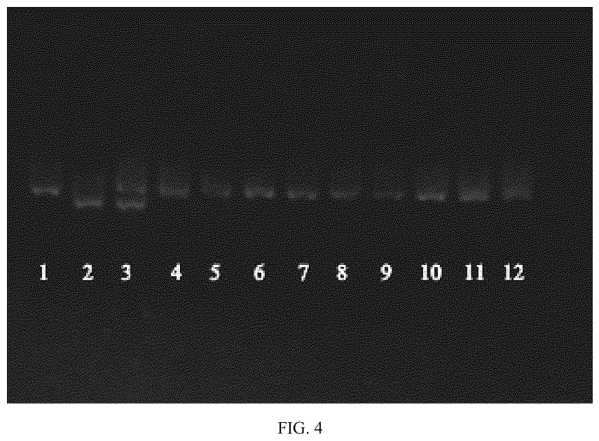

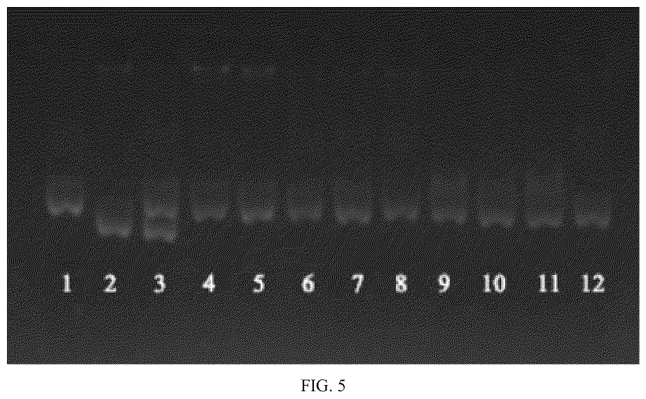

PCR amplification products were detected by 4% agarose gel electrophoresis, and some results are shown in FIG. 4 and FIG. 5.

The electrophoretic detection band patterns were analyzed. Among them, if the band tends to the parent HZ, it indicates that the resistance of this strain rice to whitebacked planthopper is better, and if the band tends to Nekken 2, it indicates that the resistance is poor.

The resistance of the tested strains to whitebacked planthopper was compared with the results predicted by the band pattern analysis, and the predicted results were consistent with the actual detection results.

Embodiment 3 Application of QTL Related to Resistance of Rice to Whitebacked Planthopper in Rice Breeding

The male parent Nipponbare, a rice variety with weak resistance, was crossed with the female parent HZ to obtain the corresponding F1, which was backcrossed with Nipponbare as the recurrent parent to BC₃F₁ generation. DNA of some individual plants of BC₃F₁ generation was extracted, and then amplified by PCR with primers of Indel Wbph-1 and Indel Wbph-2, and detected by electrophoresis.

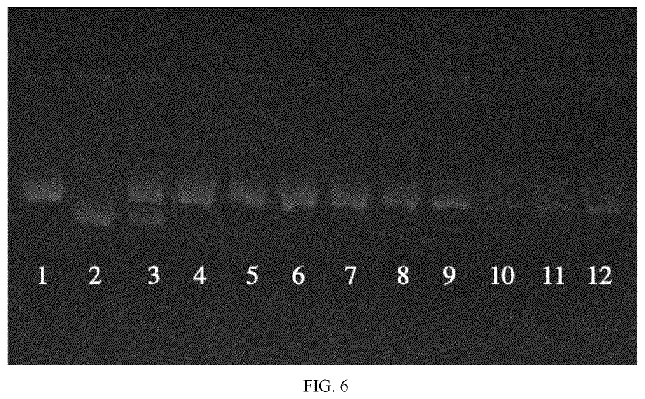

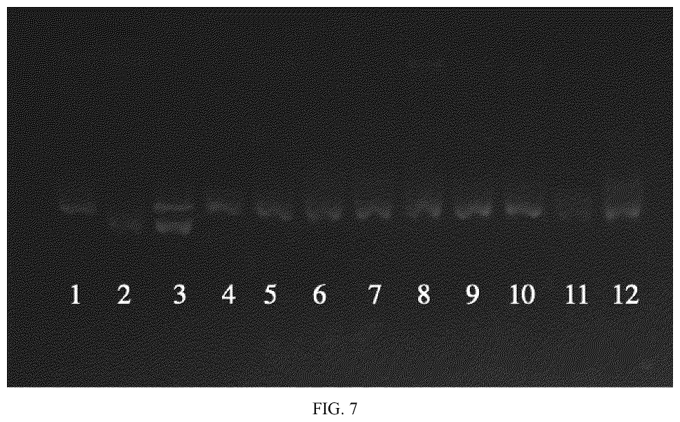

The electrophoretic detection band patterns were analyzed. The band tended to the parent HZ, which indicated that this strain rice has better resistance to whitebacked planthopper. The results are shown in FIG. 6 and FIG. 7. Using this method for screening and directional selection, the rice with better resistance to whitebacked planthopper and retaining the excellent traits of Nipponbare was obtained, which greatly improved breeding efficiency.

In conclusion, the main QTL for regulating the resistance of rice to whitebacked planthopper can effectively speed up the process of optimizing rice varieties. In the process of rice molecular assisted breeding, rice with better resistance to whitebacked planthopper can be cultivated, and the quality and yield of rice can be optimized at the same time. This method is simple, safe and effective, beneficial to improving the economic value of rice varieties, taking into account economic and ecological benefits, and suitable for large-scale popularization and application.

Various embodiments in the present specification are described in a progressive manner, and the emphasizing description of each embodiment is different from the other embodiments. The same and similar parts of various embodiments can be referred to for each other.

The above description of the disclosed embodiments enables those skilled in the art to realize or use the present disclosure. Many modifications to these embodiments will be apparent to those skilled in the art. The general principle defined herein can be realized in other embodiments without departing from the spirit or scope of the present disclosure. Therefore, the present disclosure will not be limited to these embodiments shown herein, but will conform to the widest scope consistent with the principle and novel features disclosed herein.

What is claimed is:

1. A molecular marker linked to a main QTL for regulating a resistance of a rice to a whitebacked planthopper, wherein the molecular marker comprises Indel Wbph-1 and Indel Wbph-2;
wherein, primer pairs for the Indel Wbph-1 are:
upstream primer of the Indel Wbph-1:
SEQ ID NO: 1
5′-AGGAGACCGCCGTTTTTGAT-3′,;
downstream primer of the Indel Wbph-1:
SEQ ID NO: 2
5′-AAGCGTTTCGTTTCGGTTGG-3′,;
primer pairs for the Indel Wbph-2 are:
upstream primer of the Indel Wbph-2:
SEQ ID NO: 3
5′-CTATGGCTACCGCGACGTAC-3′,;
downstream primer of the Indel Wbph-2:
SEQ ID NO: 4
5′-GCTGCTCACGTCGACGAC-3′,.

2. A method of an application of the molecular markers linked to the main QTL for regulating the resistance of the rice to the whitebacked planthopper of claim 1 in a selection and a breeding of rice varieties resistant to the whitebacked planthopper.

3. A method for a selection and a breeding of rice varieties resistant to the whitebacked planthopper, comprising:
extracting a rice DNA to obtain a DNA template, using the primer pairs of the Indel Wbph-1 of claim 1 for a first PCR amplification of the DNA template to obtain first amplification products, carrying out a first electrophoresis detection on the first amplification products to obtain a first band pattern, and using the primer pairs of the Indel Wbph-2 of claim 1 for a second PCR amplification of the DNA template to obtain second amplification products, carrying out a second electrophoresis detection on the second amplification products to obtain a second band pattern, and analyzing the resistance of the rice to the whitebacked planthopper by the first band pattern and the second band pattern.

4. The method for the selection and the breeding of the rice varieties resistant to the whitebacked planthopper of claim 3, wherein a reaction system of the first PCR amplification is: 1 μL of the upstream primer of the Indel Wbph-1, 1 μL of the downstream primer of the Indel Wbph-1, 2 μL of the DNA template, 6 μL of a mix enzyme, 1 μL of ddH₂O; and a reaction system of the second PCR amplification is: 1 μL of the upstream primer of the Indel Wbph-2, 1 μL of the downstream primer of the Indel Wbph-2, 2 μL of the DNA template, 6 μL of the mix enzyme, 1 μL of ddH₂O; and a reaction procedure of the PCR amplification comprises:
a pre-denaturation at 94° C. for 3 min; an amplification for 38 cycles by a denaturation at 94° C. for 30 s, an annealing at 57° C. for 30 s, and an extension at 72° C. for 30 s; and finally, a final extension at 72° C. for 10 min.

5. A kit for a selection and a breeding of rice varieties resistant to the whitebacked planthopper, wherein the kit for the selection and the breeding of the rice varieties resistant to the whitebacked planthopper comprises the primer pairs of the Indel Wbph-1 and the primer pairs of the Indel Wbph-2 of claim 1.
